(19) United States
(12) Patent Application Publication
(10) Pub. No.: US 2024/0165345 A1
(43) Pub. Date: May 23, 2024

(54) RECEPTACLE FOR SINGLE-HANDEDLY UNCAPPING, RECAPPING AND DISPOSING A SYRINGE'S CAP AND NEEDLE

(71) Applicant: DECAP RESEARCH AND DEVELOPMENT INCORPORATED, Richmond (CA)

(72) Inventors: Jamie MAGRILL, Richmond (CA); Ina NA, Vancouver (CA); Pnina HaRimon GROSSMAN, Hashmonaim (IL); Leon Trumpeldor KALANTARO, Netanya (IL)

(73) Assignee: DECAP RESEARCH AND DEVELOPMENT INCORPORATED, Richmond, BC (CA)

(21) Appl. No.: 18/283,649

(22) PCT Filed: Mar. 17, 2022
(86) PCT No.: PCT/CA2022/050408

§ 371 Date: Sep. 22, 2023

Related U.S. Application Data
(60) Provisional application No. 63/165,660, filed on Mar. 24, 2021.

(51) Int. Cl. A61M 5/32

(52) U.S. Cl. CPC A61M 5/3205; A61M 5/3204

(57) ABSTRACT

A receptacle for single-handedly uncapping, recapping and disposing a syringe's needle cap and needle head. The receptacle may include a receptacle unit with first and second ends and a base connected to the second end of the receptacle unit. The receptacle unit may, from its first end to its said second end, a hollow opening. The receptacle unit may, from its first end to its second end, include a wall channel. The wall channel may, at its second end be dimensioned to snugly engage the syringe's needle cap. Additionally, the wall channel may include a device for snuggly engaging a needle cap circumferential ridge and a needle head circumferential ridge.

### FIELD OF THE INVENTION

The invention relates to the field of receptacles and, particularly, to a receptacle for single-handedly uncapping, recapping and disposing a syringe's needle cap and needle head.

### BACKGROUND OF THE INVENTION

Needlestick injuries are a serious occupational hazard for healthcare, veterinary and animal care workers. Although the wound from a needlestick is small and easily treated, needlestick injuries carry a high risk of infection and potential exposure to cytotoxic or pathogenic materials (HIV, Hepatitis, zoonotic diseases).

According to the CDC's National Institute for Occupational Safety and Health (NIOSH), an estimated 600,000 to 800,000 occupational needlesticks occur each year in healthcare workers. (Infection Control Resource, 2010). Up to 40% of all NSIs occur from uncapping or recapping needles (NIOSH Alert: Preventing Needlestick Injuries in Healthcare Settings, 2000). Cost of treatment for a needlestick injury is on average $600 USD, with an annual cost of ˜$200 million USD in North America.

Furthermore, according to data from Estimates by Medical Care Data International, accidental needlesticks account for 80% of all occupationally acquired diseases (HIV, Hepatitis, zoonotic diseases). The transmission of blood-borne diseases is an area of great concern. Studies indicate that needlestick injuries are responsible up to 66,000 new Hep B, 16,000 new Hep C and 1,000 new HIV infections each year.

Needlesticks tend to occur during a procedure (e.g. drawing blood, drawing down drugs for injection, or administering medicine), while recapping needles, when disposing of needles, and during surgery. While a steady hand, a cooperative patient and minimal distractions can contribute to an ideal setting for avoiding needlestick injuries, needlestick injuries are still prevalent in healthcare, veterinary and animal care environments. OSHA standards recommend against recapping, however it is a frequent practice in all fields where needles are used. Therefore, when recapping or uncapping needles, it is recommended to use only one hand and to keep fingers away from the needlepoint to reduce the risk of needlestick injuries.

Accordingly, a need exists for an invention which enables users to uncap, recap, and dispose of a wide range of needle caps and needles with one hand while using the other hand to hold a subject in place.

### SUMMARY OF THE INVENTION

The invention is a compact receptacle that helps users to single-handedly uncap syringes prior to use, recap them, and then safely dispose of them. The invention enables users to uncap, recap, and dispose of a wide range of needle sizes and types with one hand while using the other hand to hold a subject in place.

According to the present invention there is provided a receptacle for single-handedly uncapping, recapping and disposing a syringe's needle cap and needle head. The receptacle may include a receptacle unit with first and second ends and a base connected to the second end of the receptacle unit. The receptacle unit may, from its first end to its said second end, include a hollow opening. The receptacle unit may, from its first end to its second end, include a wall channel. The wall channel may, at its second end be dimensioned to snugly engage the syringe's needle cap. Additionally, the wall channel may include a device for snuggly engaging a needle cap circumferential ridge and a needle head circumferential ridge.

Other aspects of the invention will be appreciated by reference to the detailed description of the preferred embodiment and to the claims that follow.

### BRIEF DESCRIPTION OF THE DRAWINGS

The preferred embodiment of the invention will be described by reference to the drawings thereof in which:

FIG. 1 illustrates a standard syringe; (PRIOR ART)

FIG. 2 is a perspective view of a receptacle for single-handedly uncapping, recapping and disposing a syringe needle cap and needle, according to an embodiment of the invention;

FIG. 3 is an enlarged version of the A-A circle shown in FIG. 1;

FIG. 4 is a front elevation view of the mountable receptacle shown in FIG. 1;

FIG. 5 illustrates the steps of single-handedly uncapping, recapping and disposing a syringe needle cap and needle, using a receptacle of an embodiment of the invention, wherein:

FIG. 5A illustrates the uncapping step where the needle cap is engaged with a portion of the receptacle of FIG. 1;

FIG. 5B illustrates the uncapping step where a syringe body is removed from the needle cap;

FIG. 5C illustrates the step of disposing the needle cap where the needle cap is aligned with notches on the receptacle;

FIG. 5D illustrates the step of disposing the needle cap where the needle cap has been disposed in the receptacle;

FIG. 5E illustrates the step of disposing the needle head where the needle head is aligned with the notches on the receptacle; and

FIG. 5F illustrates the step of disposing the needle head where the needle head has been disposed in the receptacle.

### DESCRIPTION OF THE PREFERRED EMBODIMENT OF THE INVENTION

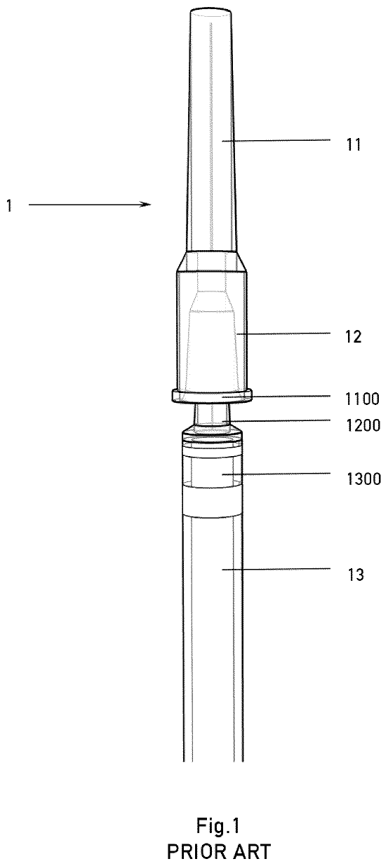

Referring to FIG. 1, a standard syringe 1 is depicted. The syringe 1 includes a needle cap 11, needle head 12, a syringe body 1300 and a syringe plunger 13 residing inside the syringe body. The cap 11 includes a circumferential cap ridge 1100 near its base and the needle head 12 includes a circumferential needle ridge 1200 also near its base.

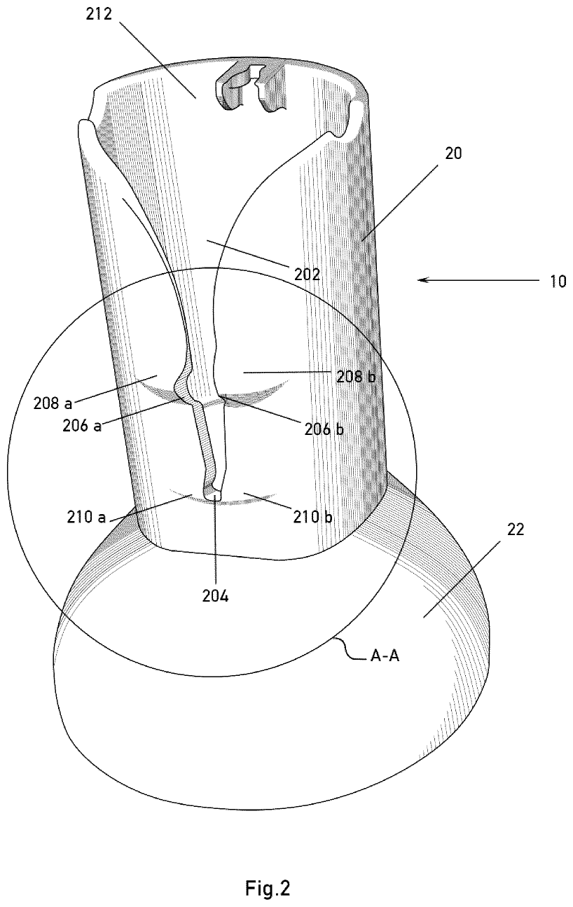

Referring to FIG. 2, a receptacle 10 for single-handedly uncapping, recapping and disposing the needle cap 11 and needle head 12 is shown to include a receptacle unit 20 with an opening 212 and a base 22. In one embodiment, the receptacle unit 20 is cylindrical and has a standard screw-track at the bottom allowing for it to be screwed onto the base 22. As those skilled in the art will appreciate, the receptacle unit 20 could take on other shapes and is not necessarily limited to a cylinder. Furthermore, the receptacle unit 20 could connect to the base 22 via other conventional means, or the receptacle unit 20 and the base 22 could be formed as one single body. Preferably, the base 22 is sufficiently wide support the receptacle unit and mounts to any flat surface, for example, via double-sided tape. The receptacle unit 20 may have a certain wall thickness. For example, in one embodiment where the receptacle unit is a cylinder, the thickness of the cylinder wall varies between 0.5 mm to approximately 7 mm, but as those skilled in the art will appreciate, the wall thickness may be modified for certain receptacles to accommodate various syringe sizes.

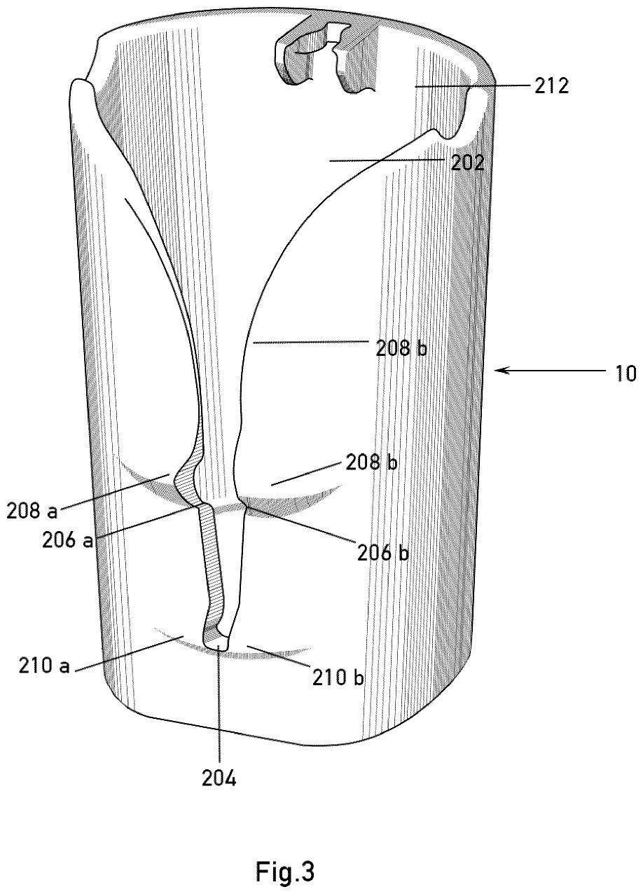

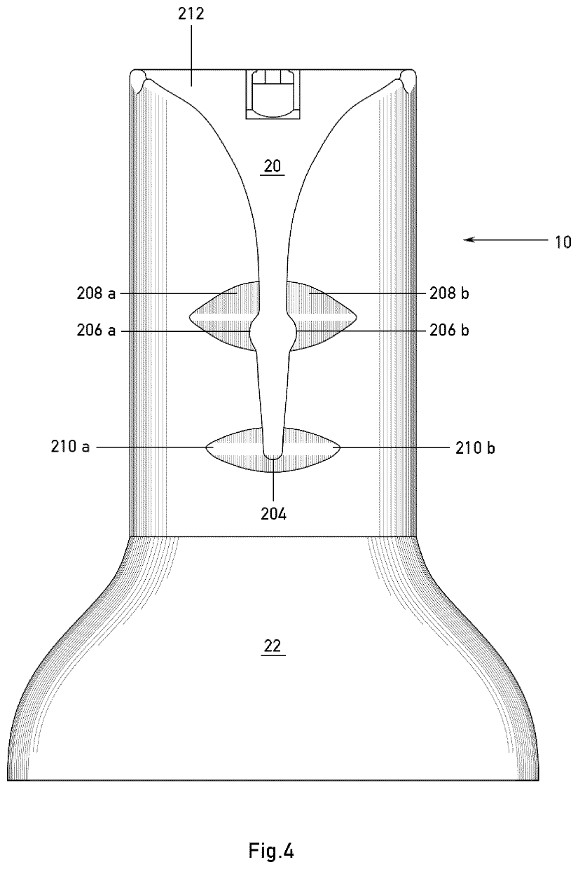

Referring to FIGS. 3 and 4, in one embodiment, the receptacle unit 20 includes a tapered wall channel 202 starting from the opening 212, narrowing down towards the base 22 and forming an end 204. The width of the wall channel 202 adjacent the end is dimensioned to snugly fit and hold the needle cap 11 for a user to uncap and recap the syringe as more fully discussed below. Preferably, the distance between the channel walls adjacent end 204 is approximately 3.6 mm, but as those skilled in the art will appreciate, the distance may be modified for certain receptacles to accommodate various syringe sizes. Midway along the wall channel 202 are two horizontally opposed notches 206a and 206b for disposing the needle cap 11 and needle head 12. The notches 206a and 206b are located closely enough to snuggly engage the circumferential cap ridge 1100 and the circumferential needle ridge 1200 as more fully discussed below. Preferably, the distance between the notches 206a and 206b is approximately 6 mm, but as those skilled in the art will appreciate, the distance may be modified for certain receptacles to accommodate various syringe sizes. As depicted, the contour of the wall channel 202 is a deep symmetrical Y-shape, but as those skilled in the art will appreciate, the overall appearance of the wall channel is not limited to as depicted. In some embodiments, the receptacle unit 20 could also include two sets of ridges 208a, 208b, 210a and 210b to allow for more ergonomic finger placement when a user pulls the syringe body 1300 away from the receptable 10.

Preferably, the receptacle 10 for single-handedly uncapping, recapping and disposing the syringe cap 11 and needle 12 is made from plastic, but as those skilled in the art will appreciate, it can be made from any suitable material.

### Operation

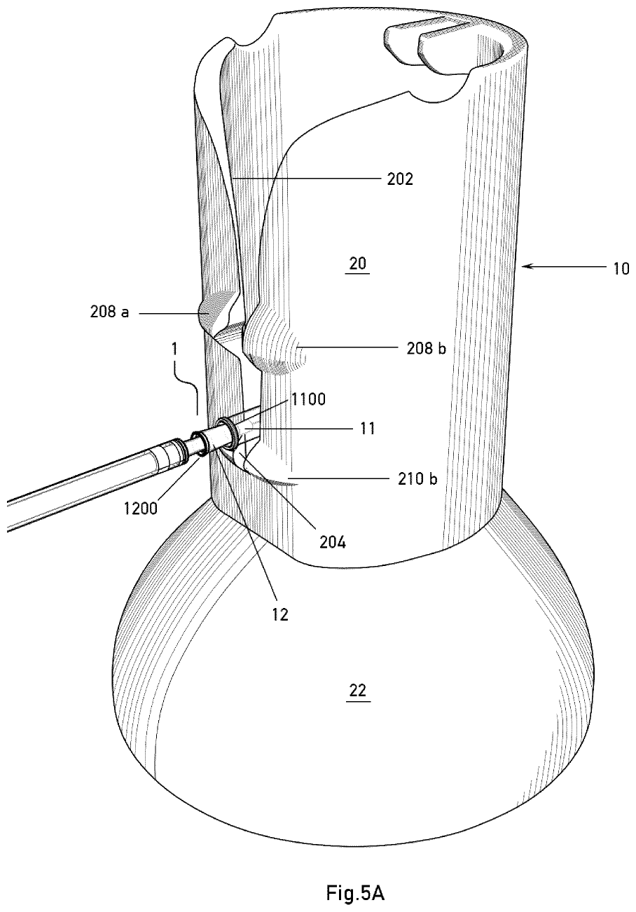

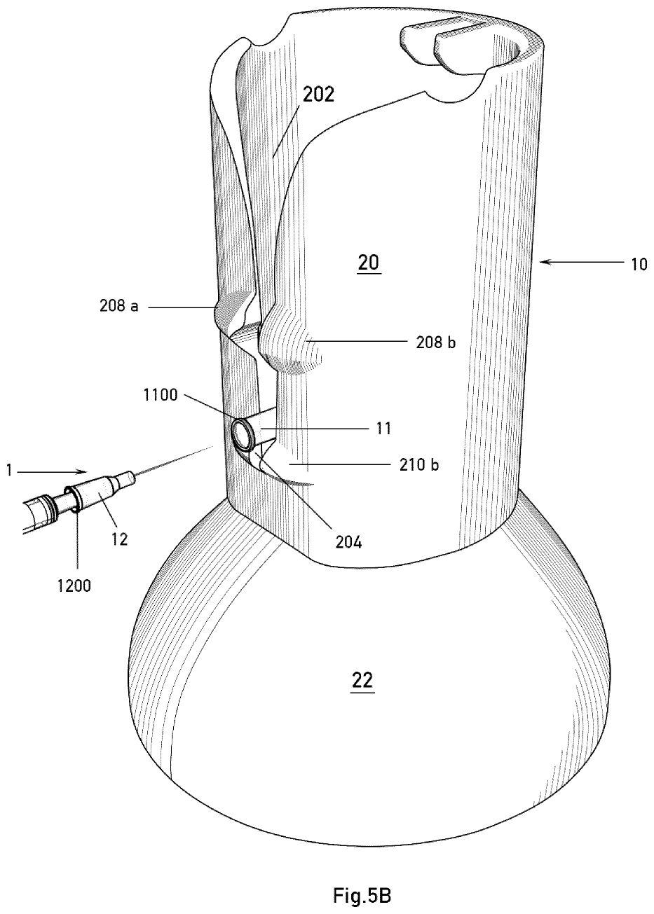

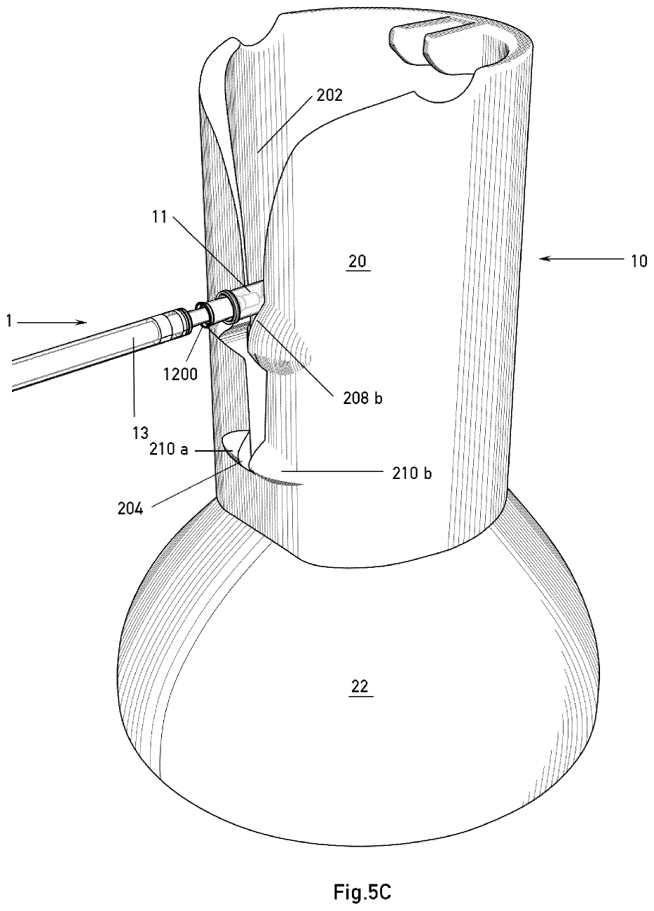

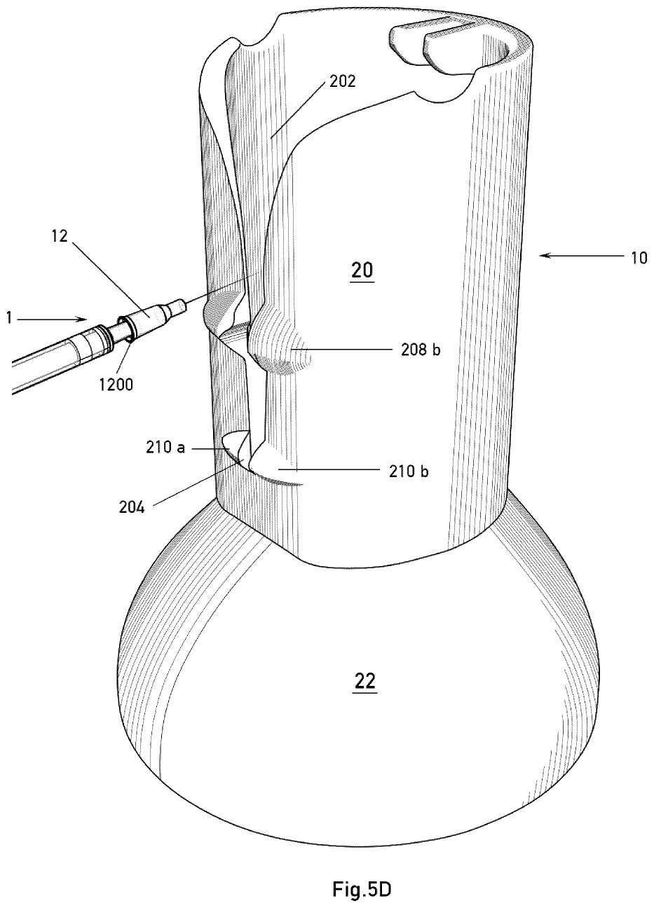

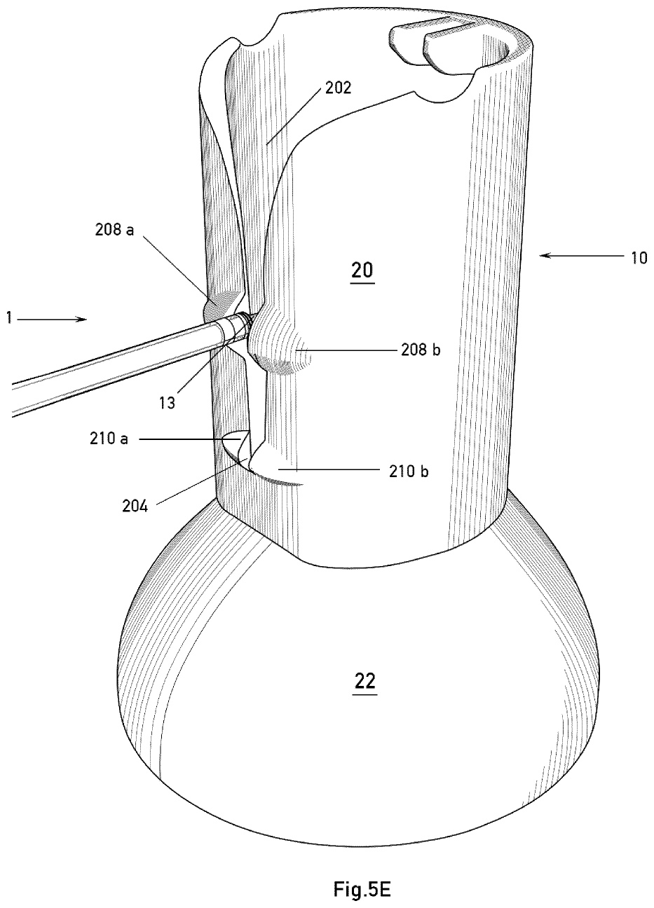

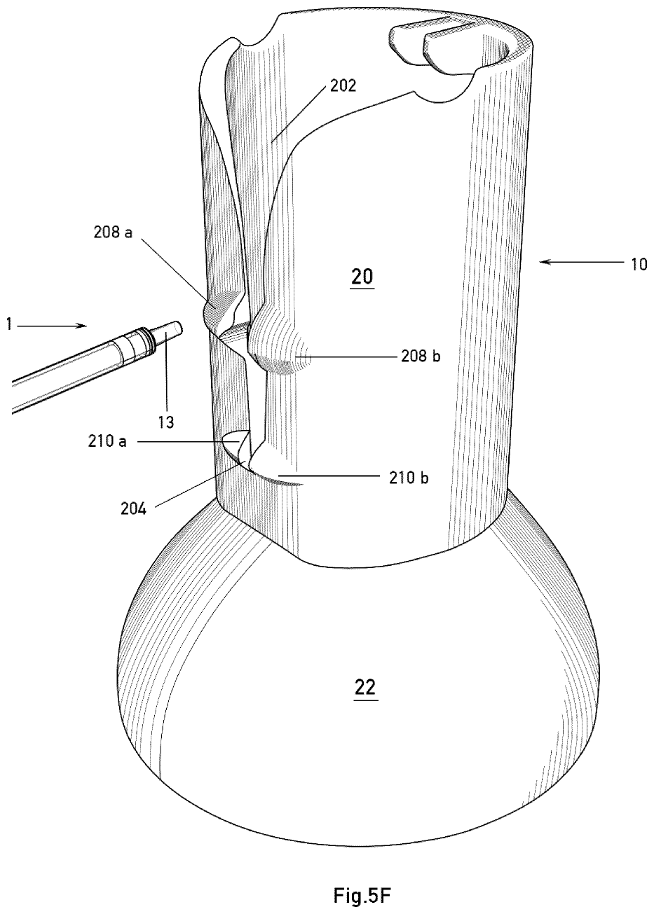

Referring to FIGS. 5A-5F, a user will single-handedly uncap the syringe 1 prior to use, recap it, and then safely dispose of the cap 11 and needle 12 while using the other hand to hold a subject in place.

Referring to FIGS. 5A-5B, to uncap and recap the syringe 1, a user will hold the syringe 1 in one hand and insert the needle cap 11 part-way into the opening 212 of the receptable unit 20. As the needle cap 11 is drawn through the wall channel 202, the tapered walls of the channel, help guide the needle cap towards the end 204. The end 204, being dimensioned to engage the needle cap 11, will frictionally fit and hold the cap 11 so that a user can simply uncap the syringe 1 by apply a horizontal pulling motion on the syringe body 1300. After a user is finished using the syringe 1, the user can recap the syringe 1 by inserting the needle head 12 back into the cap 11. To remove the syringe 1, the user will then hold the needle cap 11 with the syringe body 1300 and simply pull the syringe body and head cap together and away from the receptacle 10.

Referring to FIGS. 5C-5D, to safely dispose the needle cap 11, the user inserts the needle cap 11 into the opening 212. This time, however, the user ensures the circumferential cap ridge 1100 is along the inside of the opening 212. As the syringe 1 is drawn through the channel 202, the tapered walls of the channel, help guide the head cap 11 towards the notches 206a and 206b. Once the needle cap 11 reaches the notches 206a and 206b, the notches snuggly engage it along the circumferential cap ridge 1100. To remove the needle cap 11, the user then simply pulls the syringe body 1300 away from the receptable 10 and the needle cap 11 falls into the receptable.

Referring to FIGS. 5E-5G, to safely dispose the needle head 12, the user inserts the needle head 12 into the opening 212. This time, however, the user ensures the circumferential needle ridge 1200 is along the inside of the opening 212. As the syringe 1 is drawn through the channel 202, the tapered walls of the channel, help guide the needle head 12 towards the notches 206a and 206b. Once the needle head 12 reaches the notches 206a and 206b, the notches snuggly engage the needle head 12 along the circumferential head ridge 1200. To remove the needle head 12, the user then simply pulls the syringe body 1300 away from the receptable 10 and the needle head 12 falls into the receptable.

While embodiments of the invention have been described and illustrated, such embodiments should be considered illustrative of the invention only. The invention may include variants not described or illustrated herein in detail. Thus, the embodiments described and illustrated herein should not be considered to limit the invention as construed in accordance with the accompanying claims.

What is claimed is:

1. A receptacle for single-handedly uncapping, recapping and disposing a syringe's needle cap and needle head, comprising:
a receptacle unit with first and second ends;
a base connected to said second end of said receptacle unit;
wherein said receptacle unit from said first end to said second end comprises a hollow opening;
wherein said receptacle unit from said first end to said second end comprises a wall channel;
wherein said wall channel at said second end is dimensioned to snugly engage the syringe needle cap;
wherein said wall channel comprises means for snuggly engaging a needle cap along a circumferential ridge and a needle head along a circumferential ridge.
